(19) United States
(12) Patent Application Publication
(10) Pub. No.: US 2012/0252003 A1
(43) Pub. Date: Oct. 4, 2012

(54) Bacteria Identification by Phage Induced Impedance Fluctuation Analysis

(75) Inventors: Gabor Schmera, San Diego, CA (US); Laszlo Kish, College Station, TX (US)

(73) Assignee: United State of America, as Represented by the Secretary of the Navy

(21) Appl. No.: 13/075,250

(22) Filed: Mar. 30, 2011

(51) Int. Cl. C12Q 1/70; C12M 1/42

(52) U.S. Cl. 435/5; 435/287.1

(57) ABSTRACT

Methods for detection and identification of bacteria within a sample include the step of inserting a pair of electrodes into the sample. A first impedance across the electrodes is established with a first AC voltage source having a first frequency. A phage is introduced into the sample, and impedance fluctuations that are caused by ion release by the bacteria due to the phage introduction are measured. The use of impedance fluctuations instead of voltage fluctuations to detect and identify bacteria minimizes 1/f noise effects and increases system sensitivity. To further increase system sensitivity by eliminating thermal noise, a second impedance across the electrodes can be established using a second AC voltage source at a second frequency. Second impedance fluctuations are cross-correlated to the first impedance fluctuations, and the cross-correlation results are analyzed to determine whether or not bacteria are present in the sample based on phage electrical activity.

### FEDERALLY-SPONSORED RESEARCH AND DEVELOPMENT

This invention (Navy Case No. 100811) can be assigned to the United States Government and can be available for licensing for commercial purposes. Licensing and technical inquiries may be directed to the Office of Research and Technical Applications, Space and Naval Warfare Systems Center, Pacific, Code 72120, San Diego, Calif. 92152; voice (619)553-5118; e-mail ssc pac t2@navy.mil.

### FIELD OF THE INVENTION

The present invention applies generally to bacteria detection systems. More specifically, some embodiments of the invention pertain to systems and methods for the detection of bacteria within a sample by introducing a phage into the sample and then measuring the resulting phage-induced impedance fluctuations across the sample.

### BACKGROUND OF THE INVENTION

Fluctuation-Enhanced chemical and biological Sensing (FES) can be known in the prior art. FES can be based on stochastic analysis and simulation and utilizes the stochastic component of sensor signals that can be caused by the statistical interaction between the sample being tested and the sensor. A typical FES system utilizes specially designed sensors, advanced signal processing and pattern recognition algorithms to measure electrical fluctuations in the sample, which can be caused by ion release due to disintegration and/or dissolution of bacteria during an induced phage infestation.

Many prior art FES methods for detecting and identifying bacteria are based on the detection and analysis of direct current (DC) voltage fluctuations, which are caused by the stochastic emission of ions during phage infection of a sample. For these systems and methods, a two-electrode nano-well device can be immersed in the carrier fluid containing a phage-infected sample and the microscopic voltage fluctuations are measured across the electrodes.

However, prior art methods that measure DC voltage fluctuations can have some fairly significant disadvantages. More specifically, these methods have not been shown to work for small bacterium numbers; all experiments so far used large samples (typically on the order of 10 million bacteria per sample). This can be because these techniques measure fluctuations in the DC electrical field; i.e., the underlying and assumed phenomenon can be the separation of positive and negative ions. Second, prior art DC FES system sensitivities can be limited by the presence of strong 1/f background noise (pink noise). Additionally, drift, aging of the electrode material and dependence on surface effects and corrosion can further degrade the performance of these types of systems.

In view of the above, it can be an object of the present invention to provide systems and methods for detecting and identifying bacteria in a sample by measuring impedance fluctuations due to phage infestation of the sample. Another object of the present invention can be to provide systems and methods for detecting and identifying bacteria in a sample that offers several orders of magnitude improvement in sensitivity and higher reproducibility, at the expense of somewhat more sophisticated sensor circuitry and signal processing algorithms. Yet another object of the present invention can be to provide systems and methods for detecting and identifying bacteria in a sample that use alternating current (AC) impedance, so that the systems and methods work even when the negative and positive ions in the sample are in balance. Still another object of the present invention can be to provide systems and methods for detecting and identifying bacteria in a sample that increases detection sensitivity by minimizing the effect of noise sources such as 1/f noise, thermal noise and amplifier noise.

### SUMMARY OF THE INVENTION

Methods and systems for accomplishing the methods for the detection and identification of bacteria within a sample according to several embodiments of the present invention can include the initial step of inserting a pair of electrodes into said sample so that the electrodes are in contact with the sample (Alternatively, the sample could be place in contact with structure containing the electrodes. Additionally, sensor other than electrodes could be used, provided the sensors can detect and measure impedances). The methods and systems can further include the step of establishing a first impedance across the electrodes with a first alternating current (AC) voltage source, with the first AC source having a first frequency (f₁).

The methods and systems can further include introducing a phage into the sample. As the phage causes the disintegration and/or dissolution of bacteria (if any) in the sample, the methods and systems can measure the impedance fluctuations of the sample which are caused by ion release by the bacteria during the phage infestation. The measurement of the impedance fluctuations can be used to determine if bacteria can be present in the sample. One way to do this could be to compare the impedance fluctuation pattern to a reference impedance fluctuation pattern of the sample, which was taken when the sample was known to be bacteria free.

To measure the resulting impedance fluctuations, the systems and methods can include the use of a lock-in amplifier that can be connected to the electrodes. A pattern generator can be connected to the lock-in amplifier, and a pattern recognizer can be connected to the pattern generator. The pattern generator and recognizer can include processors that have a non-transitory medium that contains instructions for carrying out the methods of the present invention, according to several embodiments. The pattern recognizer can have access to a database of previously recorded impedance fluctuation patters that were measured and generated from known samples.

The use of an AC source at a relatively high frequency (f₁≈10 kHz) and measurement of impedance fluctuations across the sample can allow for much greater sensitivity for the methods by avoiding the 1/f noise at the electrode surfaces. To further increase the sensitivity by avoiding thermal noise, the methods (and systems for accomplishing the methods) can include the step of establishing a second impedance across said electrodes with a second AC voltage source having a second frequency (f₂). This can establish a second impedance fluctuation across the electrodes. In several embodiments, the second impedance fluctuations can be measured and cross-correlated to the first impedance fluctuations resulting from application of the first AC voltage. The cross-correlation results can used to generate impedance fluctuation patterns that can be further analyzed to determine whether or not bacteria can be present in the sample based on phage electrical activity. Some representative phages that can be used in the systems and methods presented herein can include the T5 and Ur-λ phages.

### BRIEF DESCRIPTION OF THE DRAWINGS

The novel features of the present invention will be best understood from the accompanying drawings, taken in conjunction with the accompanying description, in which similarly-referenced characters refer to similarly referenced parts, and in which:

FIG. 1 can be an exemplary system for detecting and identifying bacteria in a sample using phage induced impedance analysis, according to several embodiments of the present invention;

FIG. 2 can be the same system as FIG. 3, but with the sample electrodes in a bridge arrangement according to several embodiments;

FIG. 3 can be a continuation of the system illustrated in FIGS. 1 and 2 from connection points A and B;

FIG. 4 can be an alternative embodiment of the portion of the system shown in FIG. 3, which further illustrates how two alternating current (AC) voltage sources at frequencies f₁ and f₂ can be used to practice the methods according to several embodiments;

FIG. 5 can be a graph that depicts the performance of the system and methods of the present invention according to several embodiments, when compared to prior art direct current (DC) systems; and

FIG. 6 can be a flowchart outlining an exemplary process for accomplishing the methods of detecting and identifying bacteria in a sample, according to several embodiments of the present invention.

### DETAILED DESCRIPTION OF THE EMBODIMENTS

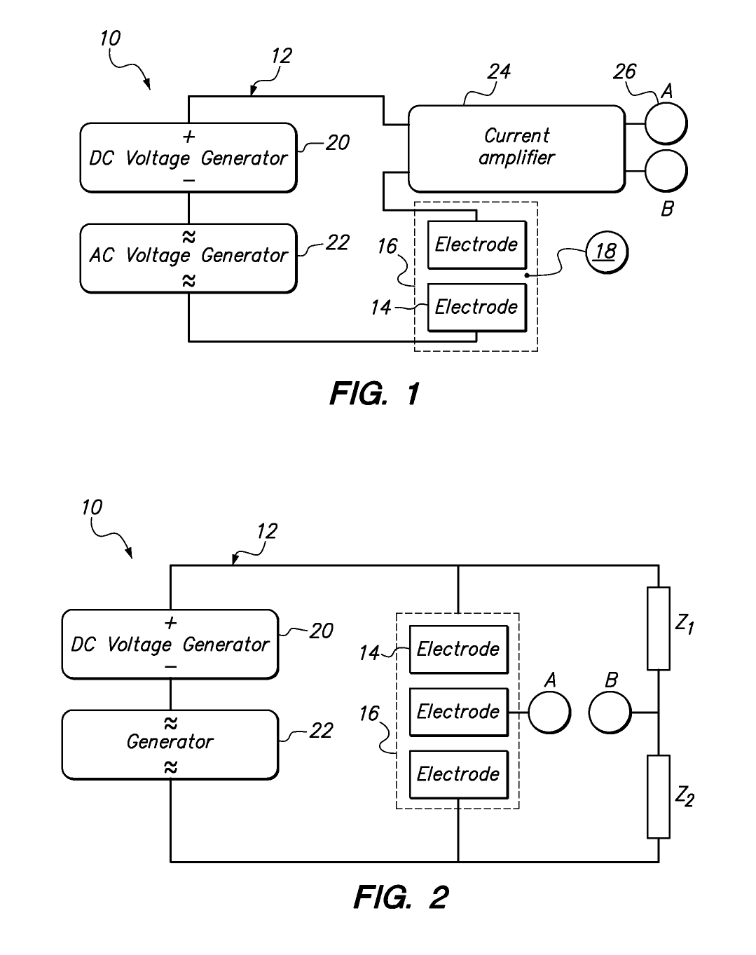

Referring now to the Figures, FIG. 1 illustrates the system 10 for identifying bacteria in a sample using phage induced impedance fluctuation analysis, according to several embodiments of the present invention. As shown in FIG. 1, the system 10 can include an electrical circuit 12. Circuit 12 can include a pair of electrodes 14 that are inserted into the sample 16 to be tested. A phage 18 can be introduced into the sample 16. As the phage interacts with the bacteria, the disintegration/dissolution of the bacteria (if any) in the sample creates electrical activity. That electrical activity can be measured and interpreted to determine whether (or not) there can be bacteria in the sample 16. The manner in which this can be accomplished, as well as the structure of circuit 12, can be described more completely below.

As shown in FIG. 1, circuit 12 can include a direct current (DC) voltage generator 20. The role of the DC voltage generator 20 can be to apply a DC voltage across electrodes 14 to attract the bacteria to one of the electrodes 14. The system 10 of the present invention can further include an alternating current (AC) voltage generator 22. AC generator 22 can be used to apply an AC voltage across circuit 12, which can allow for AC impedance fluctuation measurements across electrodes 14. With this configuration, the interference from 1/f noise in the electrical Coulomb field at the surfaces of electrodes 14 can be avoided by using an AC voltage source having a relatively high probing frequency (such as 10 KHz). The 1/f noise, which can be caused by the DC potential fluctuations in the vicinity of the electrodes, can be the primary sensitivity limiting factor for DC systems and methods that are known in the prior art.

FIG. 1 illustrates a relatively simple realization of the systems according to several embodiments of the present invention, with two electrodes 14 inserted into sample 16. With this configuration, fluctuations in impedance across electrodes are measured. In several alternative embodiments, however, and as shown in FIG. 2, the electrodes 14 can be arranged within circuit 12 and inserted into sample 16 in a bridge arrangement. In some embodiments, similar arrangements with more than three electrodes are possible.

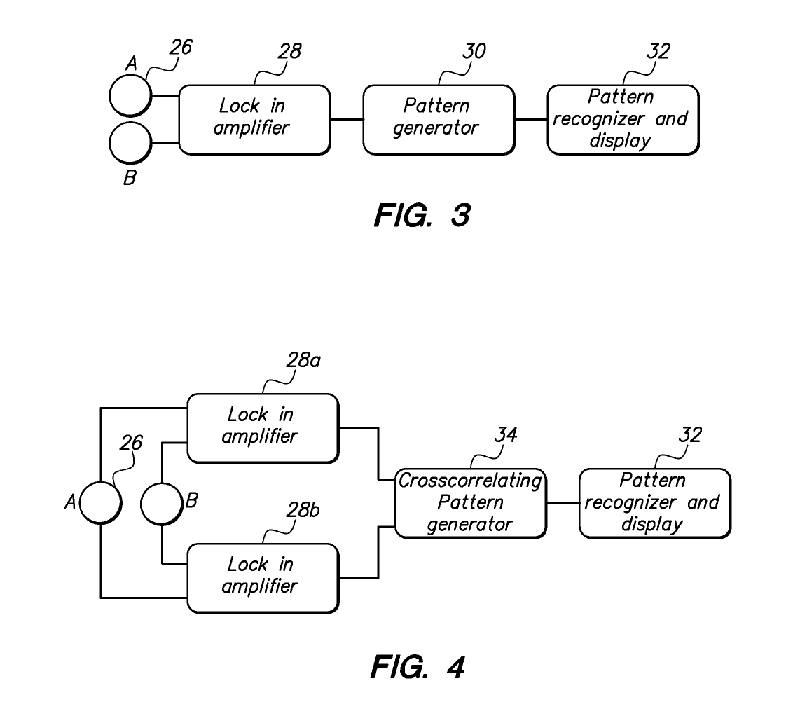

The impedance fluctuations across the electrodes 14 can be measured using additional components that are connected to connection points 26 in FIGS. 1 and 2. To facilitate the measurement of the impedance fluctuation, a current amplifier 24 can be included in circuit 12. Next, and as shown in FIG. 3, connection points 26 can be connected to the differential input of a lock-in amplifier 28. Stated differently, amplifier 24 can interconnect electrodes 14 and lock-in amplifier 28 in circuit 12. Lock-in amplifier 28 can further be connected to a pattern generator 30 (such as a spectrum analyzer, for example), and a pattern recognizer 32 can be connected to pattern generator 30. Pattern generator 30 and pattern recognizer 32 can include processors that have non-transitory computer readable medium. The computer readable medium can contain computer instructions for accomplishing the methods according to several embodiments of the present invention. Pattern recognizer 32 can further have access to a database and/or data store (not shown in the Figures) of previously recorded impedance fluctuation patterns that were measured and generated from known samples. The lock-in amplifier 28, pattern generator 30 and pattern recognizer 32 can be driven by the same AC voltage generator 22 that can be connected to the electrodes 14.

As stated above, utilizing the AC voltage generator 22 and measuring the conductance fluctuations across the electrodes 14 can result in a significantly higher sensitivity compared to the prior-art methods where DC field fluctuations are measured. By properly setting the time-constant of the lock-in amplifier 28, its output will provide a slowly fluctuating AC signal that is proportional to the low-frequency conductance fluctuations of the sample 16, which are due to electrical activity caused by the introduction of phage 18 into sample 16.

In order to further improve the performance of the system, it can be desirable to reduce the interference caused by thermal noise and by the amplifier noise. This can be accomplished by establishing a second AC voltage across the circuit 12. To do this, a second AC source 22 (not shown) can be connected to circuit 12 at a different frequency f₂ than frequency f₁. Additionally, and referring now to FIG. 4, an additional lock-in amplifier 28b can be attached to connection points 26 and synchronized to frequency f₂. Cross-correlating pattern generator 34 (for example, a cross-spectrum analyzer) can be connected to lock-in amplifiers 28 and the aforementioned pattern recognizer 32 can be connected to cross-correlating pattern generator 34. By using two separate frequencies, a sufficiently large AC drive current and cross correlation measurements, the thermal noise and amplifier noise can also be reduced. By fine-tuning these system parameters, detecting and identifying a single infected bacterium becomes a possibility.

In order to quantitatively estimate the improvement in sensitivity by the systems and methods according to several embodiments, an analysis and comparison of the signal strengths produced by the methods according to several embodiments and the DC methods described in the prior art can be disclosed. It can be seen how the presence of 1/f noise (and thermal noise) limits the sensitivity of both systems.

The DC methods of the prior art can be based on a concentration cell (two electrodes of identical metals with fluctuating electrolyte concentration). The voltage U_{cc} generated by a concentration cell can be described by the Nernst equation:

$${U}_{cc} = \frac{kT}{Zq} ln \frac{{n}_{2}}{{n}_{1}} \qquad ( 1 )$$

where k can be the Boltzmann constant, T can be the absolute temperature, Z can be the valence number of the ions, q can be the charge of an electron, and n₁ and n₂ are the ion concentrations in the vicinity of the electrodes. At room temperature, Eq. 1 reduces to:

$${U}_{cc} = \frac{0.26}{Z} ln \frac{{n}_{2}}{{n}_{1}} [ Volt ] \qquad ( 2 )$$

Now let n₂=n₁+Δn represent the change in concentration at an electrode 14 that can be caused by an infestation of phage 16. Assuming small relative concentration change, |Δn|<<n₁, the observed voltage fluctuation during DC measurements in the prior art can be:

$$Δ {U}_{sep} = \frac{kT}{Zq} ln ( \frac{{n}_{1} + Δ n}{{n}_{1}} ) = \frac{kT}{Zq} ln ( 1 + \frac{Δ n}{{n}_{1}} ) \approx \frac{kT}{Zq} \frac{Δ n}{{n}_{1}} = \frac{0.026}{Z} \frac{Δ n}{{n}_{1}} \qquad ( 3 )$$

To estimate the voltage fluctuations when using the AC methods according to several embodiments, the ion concentrations at one of electrodes 14 are used. Here too, the ion concentrations in the vicinity of the electrodes will determine the conductance and its fluctuations even under anisotropic conditions. For sake of simplicity, it can be assumed that a single AC current generator can be used; then the observed voltage fluctuations that are due to conductance fluctuations during measurement according to the systems and method of the present invention according to several embodiments can be simply:

$$Δ {U}_{bip} = {U}_{0} \frac{Δ n}{{n}_{1}} \qquad ( 4 )$$

(This analysis assumes that the electrodes 14 are approximately the same size). It can be evident from equations (3) and (4) that characteristics of the signals measured by the two methods are very similar. However, the methods of the present invention according to several embodiments produce significantly higher signal levels (and drastically reduced noise levels) for the reasons as stated below.

To measure the improvement or gain (G) in signal strength (power) by the squared ratio of the measured voltage fluctuations for the systems and methods of the present invention, over the prior art DC methods, let ΔU_{bip} represent voltage fluctuations for the systems of the present invention and let ΔUₛₑₚ represent voltage fluctuations for the DC systems and methods of the prior art:

$$G = {( \frac{Δ {U}_{bip}}{Δ {U}_{sep}} )}^{2} = {( \frac{{U}_{0} Z}{0.026} )}^{2} \qquad ( 5 )$$

As a concrete example, consider magnesium ions (Z=2) and 1 V effective AC voltage (U₀=1.41V) drop between electrodes 14 (this value can be proven to give Ohmic response with electrolytes); then the gain obtained is:

$$G = {( \frac{Δ {U}_{bip}}{Δ {U}_{sep}} )}^{2} = {( \frac{1.41 * 2}{0.026} )}^{2} > 11700 \qquad ( 6 )$$

Thus, the signal power using the AC methods of the present invention according to several embodiment can increase the system 10 sensitivity by four orders of magnitude over the DC systems of the prior art.

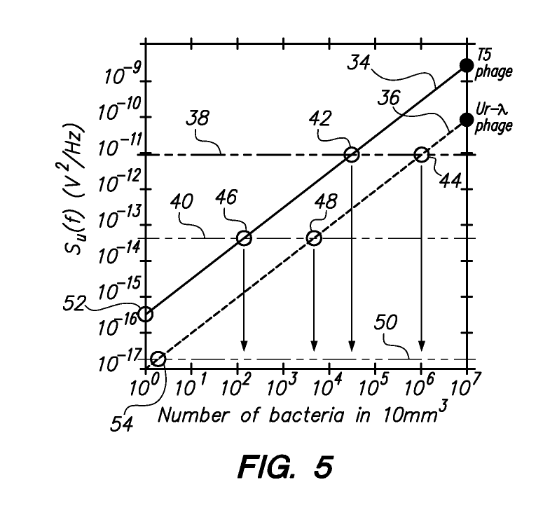

FIG. 5 can be a graph of voltage sensitivity versus the number of bacteria required for detection, which can be used to illustrate the increase in sensitivity for the systems and methods of the present invention according to several embodiments. FIG. 5 shows the measured power spectrum response for detecting E. coli bacteria using two different types of bacteriophages (phages), T5 and Ur-λ. The response for the T5 phage can be indicated by line 34, and the line representing the Ur-λphage response can be indicated by line 36 in FIG. 5. Line 38 in FIG. 5 depicts a system limitation due to 1/f noise, i.e., where the 1/f noise can be the limiting factor. For a DC system of the prior art that does not mitigate 1/f noise, and for a bacteria with a linear response, the sensitivity limit of a prior art DC system can be estimated ˜30,000 bacteria using T5 phages (point 42 in FIG. 5) and ˜1 million bacteria using Ur-λphages (point 44 in FIG. 5).

It can also be seen from FIG. 5 that when 1/f noise can be mitigated according to the systems and methods of the present invention according to several embodiments, the sensitivity of the system increases to the thermal noise threshold, which is indicated by line 40 in FIG. 5. When this occurs, the sensitivity limit can be ˜140 bacteria using T5 phages (point 46 in FIG. 5) and ˜5,000 bacteria using Ur-λphages (point 48 in FIG. 5). Thus, the systems and methods according to several embodiments can improve sensitivity by three to four orders of magnitude due to the elimination of 1/f noise (both thermal and electronic components) as a limiting factor.

The detection limits for the systems and methods according to several embodiments can be further lowered when a second AC voltage source, second lock-in amplifier 28b and cross-correlating pattern generator 34 of the present invention according to several embodiments described above are used to mitigate the effects of white noise sources such as thermal noise and amplifier noise.

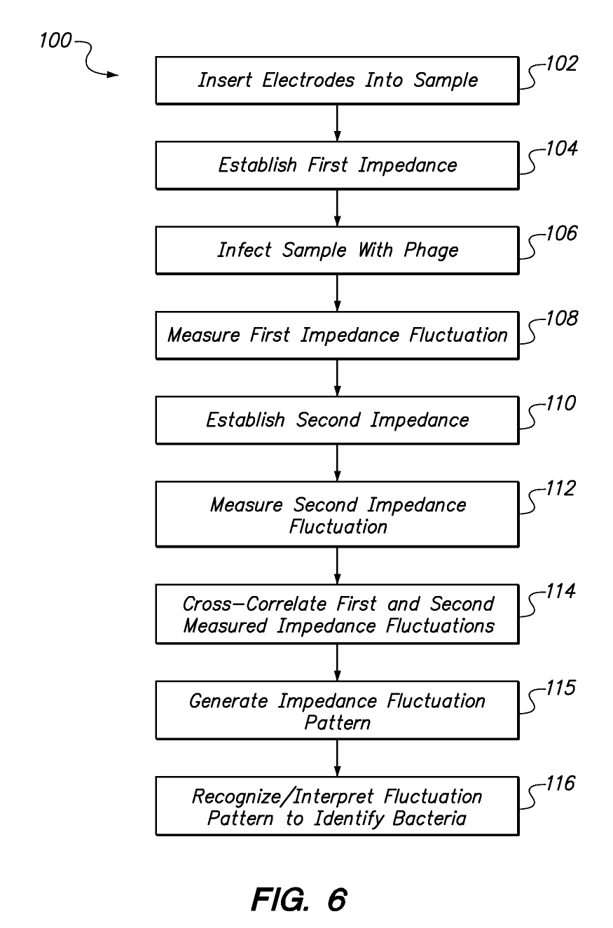

FIG. 6 can be a flowchart that can be illustrative of the methods according to several embodiments of the present invention. Method 100 in FIG. 6 can include the initial step 102 of inserting at placing two electrodes 14 in contact with sample 16. One way to accomplish this step could be to insert electrodes 14 into sample 16. The step could also be accomplished with a component other than electrodes 14, provided the component can measure impedances. This step 102 can also be accomplished with the aforementioned bridge arrangement of electrodes 14. Once the electrodes are contacting the sample, the methods can include the step of establishing a first impedance across electrodes 14, as indicated by step 104 in FIG. 6. This can be accomplished through the use of a first AC voltage source 22 at frequency f₁. Once an impedance is established, the methods can further include the step 106 of infecting sample 16 by introduction of phage 18.

As the phage 18 causes the disintegration/dissolution of the bacteria in the sample, electrical activity can be generated in the sample. The measurement of that electrical activity can be accomplished by measuring fluctuations in the first impedance, as indicated by step 108 in FIG. 6. The measured impedance fluctuations can be synchronized using a lock-in amplifier, and the resulting impedance fluctuation pattern can be input into a pattern generator 30 and pattern recognizer 32 as described above.

To further mitigate the effects of thermal noise, a second AC voltage source at f₂ can optionally be added as described above. This establishes a second impedance across electrodes 14, as illustrated by optional step 110 in FIG. 6. After the phage 18 is introduced into sample 16, a step 110 is accomplished if desired, a second impedance across electrodes 14 can be measured, as indicated by block 112. The second impedance can be synchronized at frequency f₂ using a second lock-in amplifier, as shown in FIG. 4. For these embodiments, the first and second impedances can be cross-correlated, as shown by step 114 in FIG. 6. This can be accomplished using the cross-correlating pattern generator 34 in FIG. 4. The correlated fluctuation pattern result can be sent to a pattern recognizer as described above, and for further display to the user.

Once an impedance fluctuation has been measured, an impedance fluctuation pattern can be generated by a pattern generator 30, as described above and as shown by step 115 in FIG. 6. This can occur both in embodiments where only one impedance is generated and also in the embodiment where two or more impedances are generated. For embodiments where two impedances are generated at different frequencies, a cross-correlating pattern generator 34 can be used.

Once an impedance fluctuation pattern has been generated as described above, the methods according to several embodiments can include the step of recognizing the generated impedance fluctuation patterns, as described above and depicted by step 116 in FIG. 6. This step can be accomplished by comparing the measured impedance fluctuation pattern with stored patterns in a database that correspond to impedance fluctuation patterns of known phage-infected bacteria.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value can be incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, can be intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof can be encompassed by the invention unless otherwise indicated herein or otherwise clearly contradicted by context.

What can be claimed is:

1. A method for detecting and identifying bacteria within a sample, comprising the steps of:
A) inserting a pair of electrodes into said sample;
B) establishing a first impedance across said electrodes with a first alternating current (AC) voltage source having a first frequency (f₁);
C) infecting said sample with a phage; and,
D) measuring fluctuations in said first impedance.

2. The method of claim 1 wherein said step D) further comprises the steps of:
E) measuring fluctuations in said first impedance across said sample when it is known that no bacteria are present to establish a reference impedance; and,
F) comparing the results of said step E) to said step D).

3. The method of claim 1 wherein said step C) can be accomplished with said phage being selected from the group consisting of T5 and Ur-λ.

4. The method of claim 1 wherein said first frequency can be about ten kilohertz (f₁≈10 kHz).

5. The method of claim 1, further comprising the steps of:
G) establishing a second impedance across said electrodes with a second AC voltage source having a second frequency (f₂);
H) measuring fluctuations in said second impedance across said electrodes; and,
I) cross-correlating the results of said step D) to said step H).

6. The method of claim 5, further comprising the steps of comparing the results of said step I) for said sample to the results of said step I) when it is known that no said bacteria are present in said sample.

7. A system for detecting and identifying bacteria within a sample, comprising:
at least two electrodes contacting said sample;
a first alternating current (AC) voltage source having a first frequency (f₁), said first AC voltage source establishing a first impedance across said electrodes;
a phage introduced into said sample; and,
a first means for selectively measuring said first impedance fluctuations across said at least two electrodes.

8. The system of claim 7 wherein said first frequency can be about ten kilohertz (f₁≈10 kHz).

9. The system of claim 8 wherein said first measuring means comprises a lock-in amplifier connected to said at least two electrodes, a pattern generator connected to said lock-in amplifier and a pattern recognizer connected to said pattern generator.

10. The system of claim 8 wherein said at least two electrodes comprise three electrodes connected to said first AC voltage in a bridge arrangement.

11. The system of claim 7 further comprising:
a second alternating current (AC) voltage source having a second frequency (f₂), said second AC voltage source establishing a second impedance across said electrodes;
a second means for selectively measuring said second impedance fluctuations across said electrodes; and,
a means for cross-correlating said first impedance fluctuations and said second impedance fluctuations.

12. The system of claim 11 wherein said cross-correlating means further comprises a first lock-in amplifier connected to said electrodes and synchronized to said f₁, a second lock-in amplifier connected to said electrodes and synchronized to said f₂, and a cross-spectrum analyzer connected to said first lock-in amplifier and said second lock-in amplifier.

13. The system of claim 7 wherein said phage can be selected from the group consisting of T5 and Ur-λ.
